SEQUENCE LISTING (12 sequences)

SEQ ID NO: 1 - DNA, 1041 bp, Homo sapiens
  CDS (1)..(1041)
atggatacaggccccgaccagtcctacttctccggcaatcactggttcgtcttctcggtgtaccttctcactttcctggtggggctccccctcaacctgctggccctggtggtcttcgtgggcaagctgcagcgccgcccggtggccgtggacgtgctcctgctcaacctgaccgcctcggacctgctcctgctgctgttcctgcctttccgcatggtggaggcagccaatggcatgcactggcccctgcccttcatcctctgcccactctctggattcatcttcttcaccaccatctatctcaccgccctcttcctggcagctgtgagcattgaacgcttcctgagtgtggcccacccactgtggtacaagacccggccgaggctggggcaggcaggtctggtgagtgtggcctgctggctgttggcctctgctcactgcagcgtggtctacgtcatagaattctcaggggacatctcccacagccagggcaccaatgggacctgctacctggagttccggaaggaccagctagccatcctcctgcccgtgcggctggagatggctgtggtcctctttgtggtcccgctgatcatcaccagctactgctacagccgcctggtgtggatcctcggcagagggggcagccaccgccggcagaggagggtggcggggctgttggcggccacgctgctcaacttccttgtctgctttgggccctacaacgtgtcccatgtcgtgggctatatctgcggtgaaagcccggcatggaggatctacgtgacgcttctcagcaccctgaactcctgtgtcgacccctttgtctactacttctcctcctccgggttccaagccgactttcatgagctgctgaggaggttgtgtgggctctggggccagtggcagcaggagagcagcatggagctgaaggagcagaagggaggggaggagcagagagcggaccgaccagctgaaagaaagaccagtgaacactcacagggctgtggaactggtggccaggtggcctgtgctgaaagctag

SEQ ID NO: 2 - protein, 346 aa, Homo sapiens
  TRANSMEM (18)..(41)
  TRANSMEM (52)..(73)
  TRANSMEM (88)..(111)
  TRANSMEM (132)..(153)
  TRANSMEM (188)..(212)
  TRANSMEM (229)..(250)
  TRANSMEM (259)..(278)
MDTGPDQSYFSGNHWFVFSVYLLTFLVGLPLNLLALVVFVGKLQRRPVAVDVLLLNLTASDLLLLLFLPFRMVEAANGMHWPLPFILCPLSGFIFFTTIYLTALFLAAVSIERFLSVAHPLWYKTRPRLGQAGLVSVACWLLASAHCSVVYVIEFSGDISHSQGTNGTCYLEFRKDQLAILLPVRLEMAVVLFVVPLIITSYCYSRLVWILGRGGSHRRQRRVAGLLAATLLNFLVCFGPYNVSHVVGYICGESPAWRIYVTLLSTLNSCVDPFVYYFSSSGFQADFHELLRRLCGLWGQWQQESSMELKEQKGGEEQRADRPAERKTSEHSQGCGTGGQVACAES

SEQ ID NO: 3 - DNA, 1041 bp, Homo Sapiens
  CDS (1)..(1041)
atggatacaggccccgaccagtcctacttctccggcaatcactggttcgtcttctcggtgtaccttctcactttcctggtggggctccccctcaacctgctggccctggtggtcttcgtgggcaagctgcggtgccgcccggtggccgtggacgtgctcctgctcaacctgaccgcctcggacctgctcctgctgctgttcctgcctttccgcatggtggaggcagccaatggcatgcactggcccctgcccttcatcctctgcccactctctggattcatcttcttcaccaccatctatctcaccgccctcttcctggcagctgtgagcattgaacgcttcctgagtgtggcccacccactgtggtacaagacccggccgaggctggggcaggcaggtctggtgagtgtggcctgctggctgttggcctctgctcactgcagcgtggtctacgtcatagaattctcaggggacatctcccacagccagggcaccaatgggacctgctacctggagttctggaaggaccagctagccatcctcctgcccgtgcggctggagatggctgtggtcctctttgtggtcccgctgatcatcaccagctactgctacagccgcctggtgtggatcctcggcagagggggcagccaccgccggcagaggagggtggcggggctggtggcggccacgctgctcaacttccttgtctgctttgggccctacaacgtgtcccatgtcgtgggctatatctgcggtgaaagcccggtgtggaggatctacgtgacgcttctcagcaccctgaactcctgtgtcgacccctttgtctactacttctcctcctccgggttccaagccgactttcatgagctgctgaggaggttgtgtgggctctggggccagtggcagcaggagagcagcatggagctgaaggagcagaagggaggggaggagcagagagcggaccgaccagctgaaagaaagaccagtgaacactcacagggctgtggaactggtggccaggtggcctgtgctgaaaactag

SEQ ID NO: 4 - protein, 346 aa, Homo Sapiens
MDTGPDQSYFSGNHWFVFSVYLLTFLVGLPLNLLALVVFVGKLRCRPVAVDVLLLNLTASDLLLLLFLPFRMVEAANGMHWPLPFILCPLSGFIFFTTIYLTALFLAAVSIERFLSVAHPLWYKTRPRLGQAGLVSVACWLLASAHCSVVYVIEFSGDISHSQGTNGTCYLEFWKDQLAILLPVRLEMAVVLFVVPLIITSYCYSRLVWILGRGGSHRRQRRVAGLVAATLLNFLVCFGPYNVSHVVGYICGESPVWRIYVTLLSTLNSCVDPFVYYFSSSGFQADFHELLRRLCGLWGQWQQESSMELKEQKGGEEQRADRPAERKTSEHSQGCGTGGQVACAEN

SEQ ID NO: 5 - DNA, 960 bp, Rattus sp.
  CDS (1)..(960)
atggacacaagcttctttcccggcaaccactggcttttcttttcagtggatctgttggtgttcctcgtgggactacccctcaacgtgatggccctggtggtcttcgtgaacaagctgcgtcgccgcccggtggccgtggacttacttttgcttaacctgaccatttcggacctgcttctgctcctcttcctgccattccgtatagtggaggcggcctgtggcatgaaatggattctgcccttcatcttctgccccctttctggcttccttttcttcaccaccatctacctcacctccctcttcctgatgacggtgagcatagaacgttttctgagcgtagcctacccactgtggtacaaaacccggccccggctggcccaggctggtctggtcagtggcatctgttggttcttggcatcagctcactgtagtgtgatttatgtcactgaatactggggaaatgcaacctacagccaggggaccaacggaacctgctacctggaattccgggaggaccagctggccatcctcctccccgtgcgactggaaatggctgtggtccttttcatggtgcccctgtgtattaccagttactgctacagtcgcctggtgtggattctgagccagggagccagccggcgcaggcgcaagagagtgatggggcttcttgtagccacgttgctcatcttctttgtctgcttcggcccctacaatatgtcccacgtggtgggctacgtgcgcggtgagagtccgacctggcggagctacgtgcttctcctcagcaccctcaactcttgtattgaccctctggttttctacttttcatcctccaagttccaagccgactttcatcagctcctgtctaggctgatcagagcttgtgtgccttggactcaggaagtcagcttggaactgaaggtaaagaacggagaagagccatccaaggaatgtccgagctag

SEQ ID NO: 6 - protein, 319 aa, Rattus sp.
MDTSFFPGNHWLFFSVDLLVFLVGLPLNVMALVVFVNKLRRRPVAVDLLLLNLTISDLLLLLFLPFRIVEAACGMKWILPFIFCPLSGFLFFTTIYLTSLFLMTVSIERFLSVAYPLWYKTRPRLAQAGLVSGICWFLASAHCSVIYVTEYWGNATYSQGTNGTCYLEFREDQLAILLPVRLEMAVVLFMVPLCITSYCYSRLVWILSQGASRRRRKRVMGLLVATLLIFFVCFGPYNMSHVVGYVRGESPTWRSYVLLLSTLNSCIDPLVFYFSSSKFQADFHQLLSRLIRACVPWTQEVSLELKVKNGEEPSKECPS

SEQ ID NO: 7 - DNA, 17 bp, artificial sequence
  PCR primer
cattagcatctgtgatg

SEQ ID NO: 8 - DNA, 20 bp, artificial sequence
  PCR primer
ctagctcggacactccttgg

SEQ ID NO: 9 - DNA, 20 bp, artificial sequence
  PCR primer
gccatagcactgagccaatg

SEQ ID NO: 10 - DNA, 20 bp, artificial sequence
  PCR primer
ttgtagccacgttgctcatc

SEQ ID NO: 11 - DNA, 27 bp, artificial sequence
  PCR primer
taggatccatggacacaagcttcttcc

SEQ ID NO: 12 - DNA, 29 bp, artificial sequence
  PCR primer
tactcgagctagctcggacattccttgga

(19) United States
(12) Patent Application Publication
(10) Pub. No.: US 2003/0113810 A1
(43) Pub. Date: Jun. 19, 2003

(54) Novel assay

(76) Inventors: Alan Wise, Stevenage (GB); Andrew James Brown, Stevenage (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/203,539

(22) PCT Filed: Feb. 19, 2001
(86) PCT No.: PCT/GB01/00684

(30) Foreign Application Priority Data
Feb. 18, 2000 (GB) 0003900.8
Mar. 22, 2000 (GB) 0007015.1

(51) Int. Cl. G01N 33/53; G01N 33/567; C07K 14/705

(52) U.S. Cl. 435/7.2; 530/350

(57) ABSTRACT

This invention relates to a method for identification of an agent that modulates activity of G-protein coupled receptor 41 (GPR 41), or G-protein coupled receptor 42 (GPR 42) which method comprises: (i) contacting a test agent with GPR 41, GPR42 or a variant of either thereof which is capable of coupling to a G-protein; and (ii) monitoring for GPR 41 or GPR 42 activity in the presence of a G-protein; thereby determining whether the test agent modulates GPR 41 or GPR 42 activity. An agent identifiable by this method is provided for use in the treatment of dyslipidaemia, coronary heart disease, atheroselerosis, thrombosis or obesity, angina, chronic renal failure, peripheral vascular disease, stroke, type II diabetes or metabolic syndrome (syndrome X).

### FIELD OF THE INVENTION

The present invention relates to the identification of modulators of G-protein coupled receptors, and the use of such modulators in the treatment of adipocyte associated conditions.

### BACKGROUND OF THE INVENTION

G-protein coupled receptors (GPCRs) are a super-family of membrane receptors that mediate a wide variety of biological functions. Upon binding of extracellular ligands, GPCRs interact with a specific subset of heterotrimeric G proteins that can, in their activated forms, inhibit or activate various effector enzymes and/or ion channels. All GPCRs are predicted to share a common molecular architecture consisting of seven transmembrane helices linked by alternating intracellular and extracellular loops. The extracellular receptor surface has been shown to be involved in ligand binding whereas the intracellular portions are involved in G protein recognition and activation.

Activation of receptors coupled to the Gᵢ family of G proteins leads to inhibition of adenylate cyclase and lowering of intracellular cAMP levels. In adipocytes this leads to inhibition of hormone-sensitive lipase (HSL) which regulates the process of lipolysis, i.e. the hydrolysis of triglycerides (TG) to glycerol and non-esterified fatty acids (NEFA). Inhibition of lipolysis and the concomitant lowering of NEFA levels cause a reduction of hepatic triglyceride synthesis resulting in a fall in the levels of TG-rich lipoproteins. This then leads to an elevation in high-density lipoprotein (HDL) levels, thus giving the desired clinical profile of high HDL and low TG for the treatment of dyslipidemia.

Furthermore, there are many epidemiological studies illustrating an inverse correlation between plasma HDL cholesterol and coronary artery disease. Many patients with decreased plasma HDL cholesterol levels also have elevated TG levels. Therefore an agent that inhibits adipocyte lipolysis, thereby reducing TG availability, may also result in an increase in plasma HDL cholesterol levels due to the equilibrium that exists between the levels HDL, LDL and triglycerides.

Adipocytes are known to express a number of Gᵢ-coupled receptors such as the adenosine A₁, prostaglandin EP3 and nicotinic acid receptors. Agonists at such GPCRs have been shown to be anti-lipolytic, i.e. they promote lipid lowering, and in the case of nicotinic acid have been used in the clinic to treat particular forms of dyslipidaemia. However, unlike the adenosine A₁ and EP3 receptors, the nicotinic acid receptor has yet to be identified at the molecular level.

### SUMMARY OF THE INVENTION

The present invention is based on the finding that expression of the G-protein coupled receptors GPR 41 and GPR 42 is restricted to adipose tissue. GPR 41 or GPR 42 may therefore be used as a screening target for the identification and development of novel pharmaceutical agents for use inhibiting lipolysis. Accordingly the present invention provides a method for identification of an agent that modulates GPR 41 or GPR 42 activity, which method comprises:

(i) contacting a test agent with a cell, such as an adipocyte, which expresses GPR 41, GPR 42 or a variant of either thereof which is capable of coupling to a G-protein; and

(ii) monitoring for GPR 41 or GPR 42 activity in the presence of a G-protein;

thereby determining whether the test agent modulates GPR 41 or GPR 42 activity.

The test agent may be contacted in step (i) with cells that express GPR 41, GPR 42 or a variant of either thereof. Alternatively, the test agent may be contacted in step (i) with membrane obtained from such cells. The invention also provides:

a test kit suitable for identification of an agent that modulates GPR 41 or GPR 42 activity, which kit comprises:

(a) GPR 41, GPR 42 or a variant of either thereof which is capable of coupling to a G-protein; and

(b) means for monitoring GPR 41 or GPR 42 activity.

a method for identification of an agent that inhibits lipolysis, which method comprises contacting adipocytes in vitro with a test agent which modulates GPR 41 or GPR 42 activity and which has been identified by the method of the invention and monitoring lipolysis, thereby determining whether the test substance is an inhibitor of lipolysis;

an activator of GPR 41 or GPR 42 activity or an inhibitor of lipolysis identified by a method of the invention or a polynucleotide which encodes GPR 41, GPR 42 or a variant polypeptide of either thereof, for use in a method of treatment of the human or animal body by therapy; and

use of such an activator, inhibitor or polynucleotide in the manufacture of a medicament for the treatment of dyslipidaemia and conditions associated with dyslipidaemia, coronary heart disease, atheroselerosis, thrombosis or obesity, angina, chronic renal failure, peripheral vascular disease, stroke, type II diabetes or metabolic syndrome (syndrome X).

The polynucleotide may comprise:

(a) the nucleotide sequence of SEQ ID NO: 1 or SEQ ID NO: 3,

(b) a sequence which hybridizes under stringent conditions to the complement of SEQ ID NO: 1 or SEQ ID NO: 3,

(c) a sequence that is degenerate as a result of the genetic code with respect to a sequence defined in (a) or (b), or

(d) a sequence having at least 60% identity to a sequence as defined (a), (b) or (c).

### BRIEF DESCRIPTION OF THE FIGURES

FIG. 1 illustrates the expression of GPR 41 in normal human tissues.

FIG. 2 illustrates the effect of expression of GPR 41 on the ability of acetate to stimulate GTPγS binding on membranes from HEK293T cells.

FIG. 3 illustrates the effect of transient expression of human GPR 41, on carboxylic acid-mediated stimulation of GTPγS binding in HEK293T cells.

FIG. 4 illustrates the stimulatory effect of 3-hydroxybutyrate on GTPγS binding in HEK293T cell membranes transfected to express GPR 41/G_{o1α}.

FIG. 5 illustrates the effect of transient expression of rat GPR 41 on carboxylic acid-mediated stimulation of GTPγS binding in HEK293T cells.

FIG. 6 illustrates the coupling of rat GPR 41 to yeast pheremone response pathways via G protein chimeras.

FIG. 7 illustrates the effect of various doses of propionate on rat GPR 41 expressed in Saccharomyces cerevisiae.

FIG. 8 illustrates the effect of carboxylic acid on rat GPR 41 expressed in Saccharomyces cerevisiae.

FIG. 9 illustrates the effect of 3-hydroxybutyrate on rat GPR 41 expressed in Saccharomyces cerevisiae.

FIG. 10 illustrates the effect of carboxylic acid on lipolysis in rat primary adipocytes.

FIG. 11 illustrates the effect of sodium acetate on isoprenaline-stimulated adenylate cyclase activity in rat primary adipocytes.

FIG. 12 illustrates the expression of G protein coupled receptor 42 (GPR 42) in normal human tissues.

### BRIEF DESCRIPTION OF THE SEQUENCES

SEQ ID NO: 1 shows the DNA and amino acid sequences of human GPR 41.

SEQ ID NO: 2 is the amino acid sequence alone of GPR 41. The seven transmembrane domains are identified.

SEQ ID NO: 3 shows the DNA and amino acid sequences of human GPR 42.

SEQ ID NO: 4 shows the amino acid sequence alone of GPR 42.

SEQ ID NO: 5 shows the DNA and amino acid sequence of rat GPR 41.

SEQ ID NO: 6 shows the amino acid sequence alone of rat GPR 41.

SEQ ID NO: 7 shows the sequence of PCR primer NF415.

SEQ ID NO: 8 shows the sequence of PCR primer NF416.

SEQ ID NO: 9 shows the sequence of PCR primer NF417.

SEQ ID NO: 10 shows the sequence of PCR primer NF412.

SEQ ID NO: 11 shows the sequence of PCR primer NF419.

SEQ ID NO: 12 shows the sequence of PCR primer NF420.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The present invention relates to human G-protein coupled receptors, GPR 41, GPR 42 and variants of either thereof. G-protein coupled receptors GPR 41 and GPR 42 are closely related. GPR 41 and GPR 42 have been cloned previously (Sawzdargo et al, Biochem. Biophys. Res. Commun. 239, 543-547, 1997). Sequence information for GPR 41 is provided in SEQ ID NO: 1 (nucleotide and amino acid) and in SEQ ID NO: 2 (amino acid). Similarly sequence information for GPR 42 is provided in SEQ ID NO: 3 (nucleotide and amino acid) and in SEQ ID NO: 4 (amino acid). Sequence information for rat GPR 41 is provided in SEQ ID NO: 5 (nucleotide and amino acid) and in SEQ ID NO: 6 (amino acid). The invention can therefore use polypeptides consisting essentially of the amino acid sequence of SEQ ID NO: 2, SEQ ID NO: 4, SEQ ID NO: 6 or a functional variant of either sequence. A functional chimeric receptor containing a fragment of SEQ ID NO: 2 or SEQ ID NO: 4 may therefore be used.

The term “variant” refers to a polypeptide which has the same essential character or basic biological functionality as GPR 41 or GPR 42. The essential character of GPR 41 and GPR 42 can be defined as that of a G-protein coupled receptor. Both GPR 41 and GPR 42 couple to Gᵢ-protein. Thus, the term “variant” refers in particular to a polypeptide which activates Gᵢ.

To determine whether a candidate variant has the same function as GPR 41 or GPR 42, the ability of the variant to activate Gᵢ-protein can be determined. The effect of the candidate variant on Gᵢ activation can be monitored. This can be carried out, for example, by contacting cells expressing the candidate variant with a ligand which activates Gᵢ-protein when contacted with cells that express GPR 41 or GPR 42, and measuring a Gᵢ-coupled readout. A control experiment is typically also carried out in which cells of the same type as those expressing the candidate variant, but expressing GPR 41 or GPR 42 instead, are contacted with the ligand and a corresponding Gᵢ-coupled readout is measured. The effect attained by the candidate variant can then be directly compared with that attained by GPR 41 or GPR 42.

An alternative way to determine whether a variant polypeptide has the same function as GPR 41 or GPR 42 is to determine whether the variant polypeptide binds to a ligand which activates Gᵢ when the ligand is contacted with GPR 41 or GPR 42. Thus, the ligand should activate Gᵢ when contacted with cells that express GPR 41 or GPR 42. The ability of a candidate variant to bind such a ligand can be determined directly by contacting the candidate variant with a radiolabelled ligand that binds to GPR 41 or GPR 42 and monitoring binding of the ligand to the variant. Typically, the radiolabelled ligand can be incubated with cell membranes containing the candidate variant. The membranes can then be separated from non-bound ligand and dissolved in scintillation fluid to allow the radioactivity of the membranes to be determined by scintillation counting. Non-specific binding of the candidate variant may also be determined by repeating the experiment in the presence of a saturating concentration of non-radioactive ligand. Preferably a binding curve is constructed by repeating the experiment with various concentrations of the candidate variant. The ability to bind a ligand of GPR 41 or GPR 42 may also be determined indirectly as described below.

Typically, polypeptides with more than about 65% identity, preferably at least 80% or at least 90% and particularly preferably at least 95%, at least 97% or at least 99% identity, with the amino acid sequence of SEQ ID NO: 1, 2, 3 or 4 over a region of at least 20, preferably at least 30, at least 40, at least 60 or at least 100 contiguous amino acids or over the full length of the amino acid sequence of SEQ ID NO: 1, 2, 3 or 4 are considered as GPR 41 or GPR 42 variants. The UWGCG Package provides the BESTFIT program which can be used to calculate identity (for example used on its default settings) (Devereau et al (1984) Nucleic Acid Research 12, p387-395). The PILEUP and BLAST algorithms can be used to calculate identity or line up sequences (typically on their default settings), for example as described in Algschul S. F. (1993) J. Mol. Evol. 36: 290-300; Altschul, S. F. et al (1990) J. Mol. Biol. 215: 403-10. Software for performing BLAST analyses is publicly available through the National Centre for Biotechnology Information (http://www.ncbi.nlm.nih.gov/).

Variant polypeptides therefore include naturally occurring allelic variants. An allelic variant will generally be of human or non-human mammal origin, such as bovine or porcine origin. Alternatively, a variant polypeptide can be a non-naturally occurring sequence. A non-naturally occurring variant may thus be a modified version of GPR 41 or GPR 42, i.e. a modified version of the polypeptide having the amino acid sequence of SEQ ID NO: 1,2, 3 or 4.

The amino acid sequence of GPR 41 or GPR 42 may be modified by deletion and/or substitution and/or addition of single amino acids or groups of amino acids as long as the modified polypeptide retains the capability to function as a G-protein coupled receptor. Such amino acid changes may occur in one, two or more of the intracellular domains of GPR 41 or GPR 42 and/or one, two or more of the extracellular domains of GPR 41 or GPR 42and/or one, two or more of the transmembrane domains of GPR 41 or GPR 42.

Amino acid substitutions may thus be made, for example from 1, 2, 3, 4 or 5 to 10, 20 or 30 substitutions. Conservative substitutions may be made, for example according to Table 1 below. Amino acids in the same block in the second column and preferably in the same line in the third column may be substituted for each other.

TABLE 1
Conservative amino acid substitutions

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

A variant polypeptide may be a shorter polypeptide. For example, a polypeptide of at least 20 amino acids or up to 50, 60, 70, 80, 100 or 150 amino acids in length may constitute a variant polypeptide as long as it demonstrates the functionality of GPR 41 or GPR 42. A variant polypeptide may therefore lack one, two or more intracellular domains and/or one, two or more extracellular domains and/or one, two or more transmembrane domains. A variant polypeptide may thus be a fragment of the full length polypeptide. A shortened polypeptide may comprise a ligand-binding region (N-terminal extracellular domain) and/or an effector binding region (C-terminal intracellular domain). Such fragments can be used to construct chimeric receptors preferably with another 7-transmembrane G-coupled receptor.

Variant polypeptides include polypeptides that are chemically modified, e.g. post-translationally modified. For example, such variant polypeptides may be glycosylated or comprise modified amino acid residues. They may also be modified by the addition of histidine residues, for example 6 or 8 His residues, or an epitope tag, for example a T7, HA, myc or flag tag, to assist their purification or detection. They may be modified by the addition of a signal sequence to promote insertion into the cell membrane.

The invention also utilises nucleotide sequences that encode GPR 41, GPR 42 or variants of either thereof as well as nucleotide sequences which are complementary thereto. The nucleotide sequence may be RNA or DNA including genomic DNA, synthetic DNA or cDNA. Preferably the nucleotide sequence is a DNA sequence and most preferably, a cDNA sequence. Nucleotide sequence information is provided in SEQ ID NO: 1 and SEQ ID NO: 3. Such nucleotides can be isolated from human cells or synthesised according to methods well known in the art, as described by way of example in Sambrook et al, Molecular Cloning: A Laboratory Manual, 2nd edition, Cold Spring Harbour Laboratory Press, 1989. Typically a useful polynucleotide comprises a contiguous sequence of nucleotides which is capable of hybridising under selective conditions to the coding sequence or the complement of the coding sequence of SEQ ID NO: 1 or SEQ ID NO: 3.

A polynucleotide can hybridize to the coding sequence or the complement of the coding sequence of SEQ ID NO: 1 or SEQ ID NO: 3 at a level significantly above background. Background hybridisation may occur, for example, because of other cDNAs present in a cDNA library. The signal level generated by the interaction between a polynucleotide and the coding sequence or complement of the coding sequence of SEQ ID NO: 1 or SEQ ID NO: 3 is typically at least 10 fold, preferably at least 100 fold, as intense as interactions between other polynucleotides and the coding sequence of SEQ ID NO: 1 or SEQ ID NO: 3. The intensity of interaction may be measured, for example, by radiolabelling the probe, e.g. with ³²P. Selective hybridisation may typically be achieved using conditions of low stringency (0.3M sodium chloride and 0.03M sodium citrate at about 40° C.), medium stringency (for example, 0.3M sodium chloride and 0.03M sodium citrate at about 50° C.) or high stringency (for example, 0.03M sodium chloride and 0.003M sodium citrate at about 60° C.).

The coding sequence of SEQ ID NO: 1 or SEQ ID NO: 3 may be modified by one or more nucleotide substitutions, for example from 1, 2, 3, 4 or 5 to 10, 25, 50 or 100 substitutions. The polynucleotide of SEQ ID NO: 1 or SEQ ID NO: 3 may alternatively or additionally be modified by one or more insertions and/or deletions and/or by an extension at either or both ends. The modified polynucleotide generally encodes a polypeptide which has G-protein coupled receptor activity or inhibits the activity of GPR 41 or GPR 42. Degenerate substitutions may be made and/or substitutions may be made which would result in a conservative amino acid substitution when the modified sequence is translated, for example as shown in the Table above.

A nucleotide sequence which is capable of selectively hybridising to the complement of the DNA coding sequence of SEQ ID NO: 1 or SEQ ID NO: 3 will generally have at least 60%, at least 70%, at least 80%, at least 90%, at least 95%, at least 98% or at least 99% sequence identity to the coding sequence of SEQ ID NO: 1 or SEQ ID NO: 3 respectively, over a region of at least 20, preferably at least 30, for instance at least 40, at least 60, more preferably at least 100 contiguous nucleotides or most preferably over the full length of SEQ ID NO: 1 or SEQ ID NO: 3 respectively. Methods of measuring nucleic acid and protein homology are well known in the art. For example the UWGCG Package provides the BESTFIT program which can be used to calculate homology (Devereux et al 1984). Similarly the PILEUP and BLAST algorithms can be used to line up sequences (for example are described in Altschul 1993, and Altschul et al 1990). Many different settings are possible for such programs. In accordance with the invention, the default settings may be used.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides.

Polynucleotides may be used as a primer, eg a PCR primer or a primer for an alternative amplification reaction of a probe, eg labelled with a revealing label by conventional means for identifying mutations in GPR 41 or GPR 42 that may be implicated in diseases resulting from abnormal lipolysis. Fragments of polynucleotides may be fused to the coding sequence of other proteins, preferably other G-protein coupled receptors, to form a sequence coding for a fusion protein.

Such primers, probes and other fragments will preferably be at least 10, preferably at least 15 or at least 20, for example at least 25. at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500 nucleotides in length, or even up to a few nucleotides, such as five or ten nucleotides, short of the coding sequence of SEQ ID NO: 1 or SEQ ID NO: 3.

The polynucleotides have utility in production of GPR 41, GPR 42 or variant polypeptides, which may take place in vitro, in vivo or ex vivo. The polynucleotides may be used as therapeutic agents in their own right, in gene therapy techniques. The polynucleotides are cloned into expression vectors for these purposes. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to a person skilled in the art. By way of further example in this regard we refer to Sambrook et al.

Expression vectors comprise a polynucleotide encoding the desired polypeptide operably linked to a control sequence which is capable of providing for the expression of the coding sequence by a host cell. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be plasmid, virus or phage vectors provided with a origin of replication, optionally a promoter for the expression of the said polynucleotide and optionally a regulator of the promoter. The vectors may contain one or more selectable marker genes, for example an ampicillin resistance gene in the case of a bacterial plasmid or a resistance gene for a fungal vector. Vectors may be used in vitro, for example for the production of RNA or DNA or used to transfect or transform a host cell, for example, a mammalian host cell. The vectors may also be adapted to be used in vivo, for example in a method of gene therapy.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters include the metallothionein promoter which can be induced in response to heavy metals such as cadmium. Viral promoters such as the SV40 large T antigen promoter or adenovirus promoters may also be used. All these promoters are readily available in the art.

Mammalian promoters, such as β-actin promoters, may be used. Tissue-specific promoters, in particular adipose cell specific promoters are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

The vector may further include sequences flanking the polynucleotide which comprise sequences homologous to eukaryotic genomic sequences, preferably mammalian genomic sequences, or viral genomic sequences. This will allow the introduction of the relevant polynucleotides into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Retrovirus vectors for example may be used to stably integrate the polynucleotide into the host genome. Replication-defective adenovirus vectors by contrast remain episomal and therefore allow transient expression.

Cells are transformed or transfected with the vectors to express the GPR 41 or GPR 42 polypeptide or a variant of either thereof. Such cells may be eucaryotic or prokaryotic. They include transient or, preferably, stable higher eukaryotic cell lines such as mammalian cells or insect cells, lower eukaryotic cells such as yeast, and prokaryotic cells such as bacterial cells. Particular examples of cells which may be used to express GPR 41, GPR 42 or a variant polypeptide include mammalian HEK293T, CHO, HeLa and COS7 cells. Preferably the cell line selected will be one which is not only stable, but also allows for mature glycosylation and cell surface expression of the GPR 41 or GPR 42 polypeptide or a variant of either thereof. Cells such as adipocytes expressing the GPR 41 or GPR 42 receptor or a variant polypeptide may be used in screening assays. Expression may be achieved in transformed oocytes. The GPR 41 or GPR 42 polypeptide or a variant of either thereof may be expressed in cells such as adipose tissue of a transgenic non-human animal, preferably a rodent such as a mouse.

The present invention is concerned in particular with the use of GPR 41, GPR 42 or a functional variant in screening methods to identify agents that may act as modulators of GPR 41 or GPR 42 receptor activity and, in particular, agents that may act as modulators of lipolysis. Such modulators are useful in the treatment of dyslipidaemia, coronary artery disease, atherosclerosis, obesity and thrombosis, angina, chronic renal failure, peripheral vascular disease, stroke, type II diabetes and metabolic syndrome (syndrome X).

Any suitable form of assay may be employed to identify a modulator of GPR 41 or GPR 42 activity and/or of lipolysis. In general terms, such screening methods involve contacting GPR 41, GPR 42 or a variant polypeptide with a test compound and then determining receptor activity. G-protein activation, and especially Gᵢ-protein activation, may be determined therefore. Where a test compound affects receptor activity, its effect on lipolysis can be determined by contacting adipocytes in culture with the test compound and measuring lipolysis.

Modulator activity can be determined in vitro or in vivo by contacting cells expressing GPR 41, GPR 42 or a variant polypeptide with an agent under test and by monitoring the effect mediated by the GPR 41, GPR 42 or variant polypeptide. Thus, a test agent may be contacted with isolated cells which express GPR 41, GPR 42 or a variant polypeptide. The cells may be provided in culture. Cells may be disrupted and cell membranes isolated and used.

The GPR 41, GPR 42 or variant polypeptide may be naturally or recombinantly expressed. Preferably, an assay is carried out in vitro using cells expressing recombinant polypeptide or using membranes from such cells. Suitable eucaryotic and procaryotic cells are discussed above. Preferably adipocytes are used.

Typically, receptor activity is monitored by measuring a Gᵢ-coupled readout. Gᵢ-coupled readout can be monitored using an electrophysiological method to determine the activity of G-protein regulated Ca²⁺ or K⁺ channels or by using fluorescent dye to measure changes in intracellular Ca²⁺ levels. Other methods that can typically be used to monitor receptor activity involved measuring levels of or activity of GTPγS or cAMP.

A standard assay for measuring activation of the Gᵢ family of G proteins is the GTP_{γ}S binding assay. Agonist binding to G protein-coupled receptors promotes the exchange of GTP for GDP bound to the α subunit of coupled heterotrimeric G proteins. Binding of the poorly hydrolysable GTP analogue, [³⁵S]GTP_{γ}S, to membranes has been used extensively as a functional assay to measure agonism at a wide variety of receptors. Furthermore, the assay is largely restricted to measuring function of receptors coupled to the Gᵢ family of G proteins due to their ability to bind and hydrolyse guanine nucleotide at significantly higher rates than members of the G_{q}, Gₛ and G₁₂ families. See Wieland and Jakobs, Methods Enzymol. 237, 3-13, 1994.

Yeast assays may be used to screen for agents that modulate the activity of GPR 41, GPR 42 or variant polypeptides. A typical yeast assay involves heterologously expressing GPR 41, GPR 42 or a variant polypeptide in a modified yeast strain containing multiple reporter genes, typically FUS1-HIS3 and FUS1-lacZ, each linked to an endogenous MAPK cascade-based signal transduction pathway. This pathway is normally linked to pheromone receptors, but can be coupled to foreign receptors by replacement of the yeast G protein with yeast/mammalian G protein chimeras. Strains may also contain further gene deletions, such as deletions of SST2 and FAR1, to potentiate the assay. Ligand activation of the heterologous receptor can be monitored for example either as cell growth in the absence of histidine or with a suitable substrate such as beta-galactosidase (lacZ).

Alternatively melanophore assays may be used to screen for activators of GPR 41 or GPR 42. GPR 41, GPR 42 or a variant polypeptide can be heterologously expressed in Xenopus laevis melanophores and their activation can be measured by either melanosome dispersion or aggregation. Basically, melanosome dispersion is promoted by activation of adenylate cyclase or phospholipase C, i.e. Gₛ and G_{q} mediated signalling respectively, whereas aggregation results from activation of Gᵢ-protein resulting in inhibition of adenylate cyclase. Hence, ligand activation of the heterologous receptor can be measured simply by measuring the change in light transmittance through the cells or by imaging the cell response.

Preferably, control experiments are carried out on cells which do not express GPR 41, GPR 42 or a variant polypeptide to establish whether the observed responses are the result of activation of the GPR 41, GPR 42 or the variant polypeptide. Competitive assays may be carried out on a test substance in the presence of a known activator or antagonist of GPR 41 or GPR 42.

In vitro assay systems to measure lipolysis include cell lines that can be induced to differentiate into adipocytes such as 3T3-L1(murine) and SAOS-2(human) cells (Imamura et al, J. Biol. Chem. 274, 33691-33695, 1999; Diascro et al, J. Bone & Mineral Res. 13, 96-106, 1998). Alternatively, primary adipocytes harvested from an animal or human donor may be used.

Additional assays may thus be carried out in adipocytes. For example, the hydrolysis of triglycerides (TG) to non-esterified fatty acids (NEFA) and glycerol is performed by hormone-sensitive lipase (HSL). The activity of HSL is regulated by cAMP-dependent protein kinases. Therefore, inhibition of cAMP generation by adenylate cyclase via Gᵢ-coupled receptors (e.g. GPR 41 or GPR 42 or a variant of either thereof) results in the reduction of NEFA and glycerol levels generated by adipocytes. Chromogenic assays for both NEFA and glycerol are commercially available (Randox) and can be used to verify that pre-treatment of adipocytes with an agonist for GPR 41 or GPR 42 results in a reduction in the levels of NEFA and glycerol derived from adipocytes. In addition, assays can be performed to measure the cAMP content of adipocytes in the presence and absence of modulators for GPR 41, GPR 42 or a variant thereof in order to correlate reduction in the products of lipolysis with the activation of a Gi-coupled receptor.

A standard method for identifying lipolysis inhibitors is as follows. Adipocytes, for example approximately 100,000 in 0.5 ml, are pre-treated with an agent under test. The pre-treated adipocytes are incubated in the presence of adenosine deaminase, thereby to prevent accumulation of endogenous adenosine. Incubation can be carried out for 30 minutes at 37° C. Cells are centrifuged and buffer withdrawn from below the cell layer, heated such as at 70° C. for 10 minutes and glycerol can be assayed enzymatically. A suitable assay method is described in McGowan et al, Clin. Chem. 29, 538-543, 1983).

Suitable test substances which can be tested in the above assays include combinatorial libraries, defined chemical entities, peptide and peptide mimetics, oligonucleotides and natural product libraries, such as display (e.g. phage display libraries) and antibody products. In a preferred embodiment, the test substance is a nicotinic acid (Niacin). Assays may also be carried out using known ligands of other G-protein coupled receptors to identify ligands which act as agonists at GPR 41 or GPR 42.

Test substances may be used in an initial screen of, for example, 10 substances per reaction, and the substances of these batches which show inhibition or activation tested individually. Test substances may be used at a concentration of from 1 nM to 1000 μM, preferably from 1 μM to 100 μM, more preferably from 1 μM to 10 μM.

Agents which modulate GPR 41 or GPR 42 activity and which have been identified by assays in accordance with the invention can be used in the treatment or prophylaxis of lipid disorders which are responsive to regulation of GPR 41 or GPR 42 receptor activity. Agents which activate GPR 41 or GPR 42 receptor activity and/or which have been identified as inhibitors of lipolysis are preferred. In particular, such agents may be used in the treatment of dyslipidaemia and conditions associated with dyslipidaemia such as atherosclerosis, obesity, thrombosis or coronary artery disease, angina, chronic renal failure, peripheral vascular disease, stroke, type II diabetes, and metabolic syndrome (syndrome X).

The agents may be formulated with a pharmaceutically acceptable carrier and/or excipient as is routine in the pharmaceutical art. See for example Remington's Pharmaceutical Sciences, Mack Publishing Company, Eastern Pennsylvania 17^{th} Ed. 1985. The carrier or excipient may be an isotonic saline solution but will depend more generally upon the particular agent concerned and the route by which the agent is to be administered.

The agents may be administered by enteral or parenteral routes such as via oral, buccal, anal, pulmonary, intravenous, intra-arterial, intramuscular, intraperitoneal, topical or other appropriate administration routes. A therapeutically effective amount of a modulator is administered to a patient. The dose of a modulator may be determined according to various parameters and especially according to the substance used; the age, weight and condition of the patient to be treated; the route of administration; and the required regimen. A physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of administration. Preferably, daily dosage levels are from 5 mg to 2 g.

Alternatively agents which up-regulate GPR 41 or 42 expression or nucleic acid encoding GPR 41, GPR 42 or a variant polypeptide may be administered to the mammal. Nucleic acid, such as RNA or DNA, preferably DNA, is provided in the form of a vector, which may be expressed in the cells of a human or other mammal under treatment. Preferably such up-regulation or expression following nucleic acid administration will enhance GPR 41 or GPR 42 activity.

Nucleic acid encoding the GPR 41, GPR 42 or variant polypeptide may be administered to a human or other mammal by any available technique. For example, the nucleic acid may be introduced by injection, preferably intradermally, subcutaneously or intramuscularly. Alternatively, the nucleic acid may be delivered directly across the skin using a nucleic acid delivery device such as particle-mediated gene delivery. The nucleic acid may be administered topically to the skin, or to the mucosal surfaces for example by intranasal, oral, intravaginal, intrarectal administration.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in the range of 1 pg to 1 mg, preferably to 1 pg to 10 μg nucleic acid for particle mediated gene delivery and 10 μg to 1 mg for other routes.

Polynucleotides encoding GPR 41, GPR 42 or a variant polypeptide can also be used to identify mutation(s) in GPR 41 or GPR 42 genes which may be implicated in human disorders. Identification of such mutation(s) may be used to assist in diagnosis of dyslipidaema and conditions associated with dyslipidaemia such as, atherosclerosis, obesity, thrombosis, angina, chronic renal failure, peripheral vascular disease, stroke, type II diabetes, and metabolic syndrome (syndrome X) or other disorders or susceptibility to such disorders and in assessing the physiology of such disorders.

Antibodies (either polyclonal or preferably monoclonal antibodies, chimeric, single chain, Fab fragments) which are specific for the GPR 41 or GPR 42 polypeptide or a variant thereof can be generated. Such antibodies may for example be useful in purification, isolation or screening methods involving immunoprecipitation techniques and may be used as tools to elucidate further the function of GPR 41, GPR 42 or a variant thereof, or indeed as therapeutic agents in their own right. Such antibodies may be used to block ligand binding to the receptor. A variety of protocols for competitive binding or immunoradiometric assays to determine the specific binding capability of an antibody are well known in the art (see for example Maddox et al, J. Exp. Med. 158, 1211 etseq, 1993).

The following Examples illustrate the invention.

### EXAMPLE 1

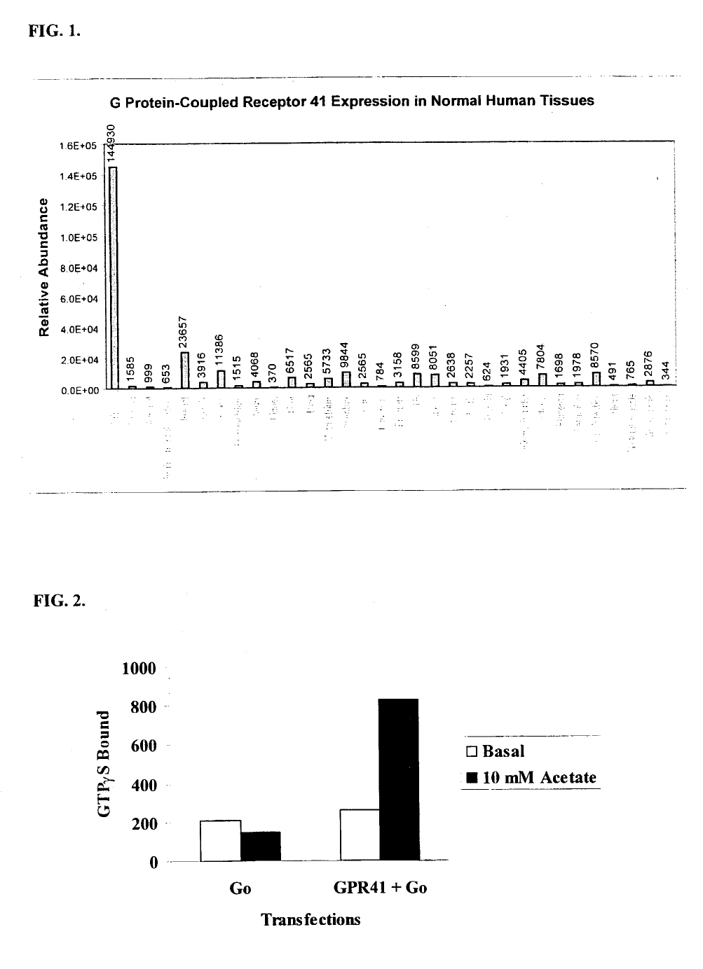

Taqman™ distribution analysis of GPR 41 and GPR 42 was carried out to study expression of GPR 41 and GPR 42 in normal human tissues. The results for GPR 41 are shown in FIG. 1; those for GPR 42 are shown in FIG. 12. These demonstrate that expression of both GPR 41 and GPR 42 is essentially restricted to adipose tissue.

### EXAMPLE 2

Mammalian cells, such as HEK293, CHO and COS7 cells, over-expressing GPR 41, GPR 42 or a variant polypeptide are generated for use in the assay. 96 and 384 well plate, high throughput screens (HTS) are employed using fluorescence based calcium indicator molecules, including but not limited to dyes such as Fura-2, Fura-Red, Fluo 3 and Fluo 4 (Molecular Probes). Secondary screening involves the same technology. Tertiary screens involve the study of modulators in rat, mouse and guinea-pig models of disease relevant to the target.

A screening assay may be conducted as follows. Mammalian cells stably over-expressing the relevant polypeptide are cultured in black wall, clear bottom, tissue culture-coated 96 or 384 well plates with a volume of 100 μl cell culture medium in each well 3 days before use in a FLIPR (Fluorescence Imaging Plate Reader—Molecular Devices). Cells are incubated with 4 μM FLUO-3AM at 30° C. in 5% CO₂ for 90 mins and are then washed once in Tyrodes buffer containing 3 mM probenecid. Basal fluorescence is determined prior to addition of agents to be tested. The GPR 41, GPR 42 or variant polypeptide is activated upon the addition of a known agonist. Activation results in an increase in intracellular calcium which can be measured directly in the FLIPR. For antagonist studies, test agents are preincubated with the cells for 4 minutes following dye loading and washing and fluorescence is measured for 4 minutes. Agonists are then added and cell fluorescence measured for a further 1 minute.

Xenopus oocyte expression may be determined as follows. Adult female Xenopus laevis (Blades Biologicals) are anaesthetised using 0.2% tricaine (3-aminobenzoic acid ethyl ester), killed and the ovaries rapidly removed. Oocytes are then de-folliculated by collagenase digestion (Sigma type I, 1.5 mg ml⁻¹) in divalent cation-free OR2 solution (82.5 mM NaCl, 2.5 mM KCl, 1.2 mM NaH₂PO₄, 5 mM HEPES; pH 7.5 at 25° C.). Single stage V and VI oocytes are transferred to ND96 solution (96 mM NaCl, 2 mM KCl, 1 mM MgCl₂, 5 mM HEPES, 2.5 mM sodium pyruvate; pH 7.5 at 25° C.) which contains 50 μg ml⁻¹ gentamycin and are stored at 18° C.

The GPR 41 or GPR 42 receptor (in pcDNA₃, Invitrogen) is linearised and transcribed to RNA using T7 (Promega Wizard kit). m′G(5′)pp(5′)GTP capped cRNA is injected into oocytes (20-50 ng per oocyte) and whole-cell currents are recorded using two-microelectrode voltage-clamp (Geneclamp amplifier, Axon instruments Inc.) 3 to 7 days post-RNA injection. Microelectrodes have a resistance of 0.5 to 2 MΩ when filled with 3M KCl.

### EXAMPLE 3

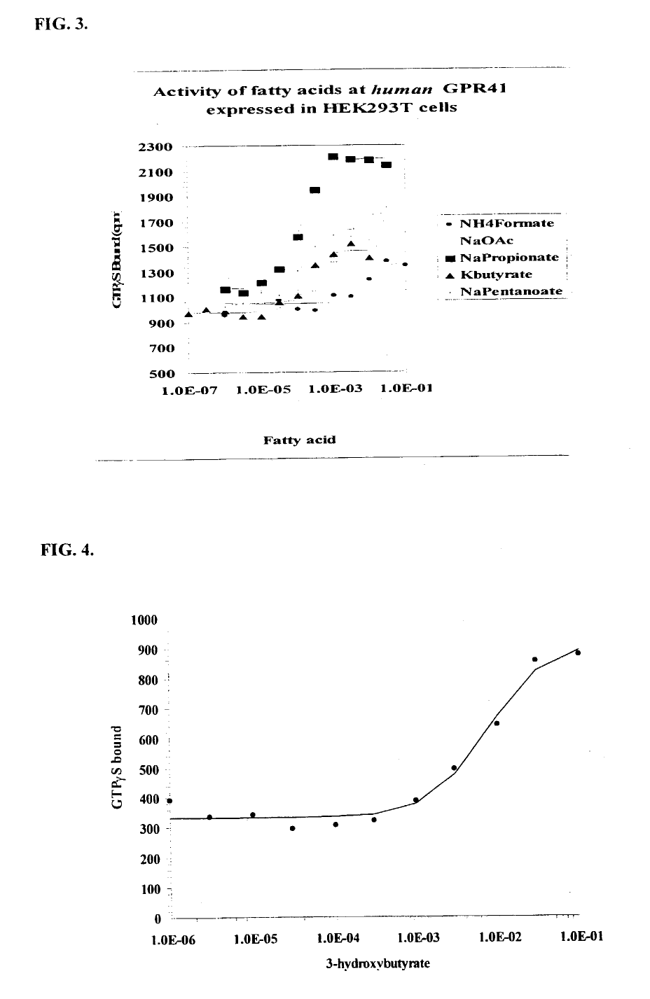

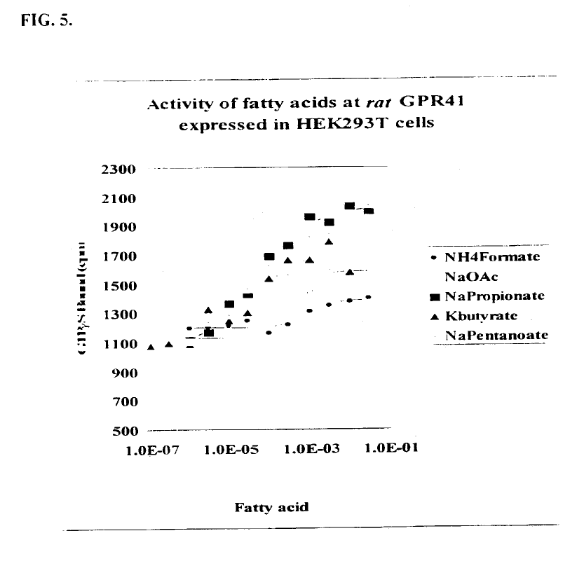

Transient transfection of the cDNA for human GPR41 together with that of the Gᵢ family G protein G_{o1α} into HEK293T cells led to acetate-mediated stimulation of [³⁵S]GTPγS binding on membranes from harvested cells (FIG. 2). Such responses were not observed in cells transfected with G_{o1α} alone. Further functional analysis demonstrated that expression of GPR41 permitted responses of differing magnitude and potency to a variety unsaturated carboxylic acids ranging from 1 carbon to 5 carbons in chain length (C1-C5) (FIG. 3), with propionate (C3) being the most potent and efficacious. In addition, the naturally occurring ketone body, 3-hydroxybutyrate, was also found to elevate [³⁵S]GTPγS binding on membranes from GPR41-expressing cells (FIG. 4). These data suggest that activation of GPR 41 by short chain carboxylic acids promotes activation Gi mediated signalling pathways, which in adipose tissue may lead to inhibition of lipolysis.

### EXAMPLE 4

The rat orthologue of human GPR41 was identified as follows. The mouse orthologue of human GPR41/42 was identified by searching public domain databases with the peptide sequence for human GPR41. The GenBank entry Accession No. AC079472 contains the high throughput draft sequence for mus musculus clone RP23-123D23. An open reading frame of 960 bp was identified (between residues 53091-52135) that was 72% homologous to the human sequence at both the DNA and protein level. This open reading frame was shorter (957 bp/319 amino acids vs 1038 bp/346 amino acids) than the corresponding human sequence. PCR primers were designed that started immediately upstream of the putative start codon (NF415 5′-CATTAGCATCTGTGATG-3′) (SEQ ID NO: 7) and that finished at the putative stop codon (NF416 5′-CTAGCTCGGACACTCCTTGG-3′) (SEQ ID NO: 8). Primers NF415 and NF416 were used to amplify the corresponding section of rat genomic DNA. This region was amplified using Pfu DNA polymerase under conditions recommended by the manufacturer (Stratagene) at an annealing temperature of 50° C. The fragment was cloned into the vector PCR-Script (Stratagene) and sequenced. The DNA sequence was 91% homologous to murine GPR41 and translated amino acid sequence 92% homologous.

Since primer NF416 encoded amino acids based on the murine sequence another section of rat DNA was amplified using primers NF417 (corresponding to the murine sequence 52 bp downstream of the stop codon 5′-GCCATAGCACTGAGCCAATG-3′) (SEQ ID NO: 9) and NF412 (5′-TTGTAGCCACGTTGCTCATC-3+ corresponding to residues 668-687 of the rat coding sequence) (SEQ ID NO: 10). These primers were used to amplify a fragment approximately 340 bp long extending beyond the putative stop codon into the 3′ untranslated region. This region was amplified using Taq DNA polymerase under conditions recommended by the manufacturer (Sigma) at an annealing temperature of 44° C. The fragment was cloned into the vector pBluescript KS (Stratagene) and sequenced. Analysis of this clone gave the 3′ sequence of rat GPR41 up to and beyond the stop codon. The DNA sequence of rat GPR41 is 70% homologous to human GPR41 and translated amino acid sequence is 71% homologous.

Primers NF419 (5′-TAGGATCCATGGACACAAGCTTCTTCC-3′) (SEQ ID NO: 11) and NF420 (5′-TACTCGAGCTAGCTCGGACATTCCTTGGA-3′) (SEQ ID NO: 12) were designed to amplify the entire coding sequence of rat GPR41 and to add flanking 5′ BamHI and 3′ XhoI restriction sites. This region was amplified using Pfu DNA polymerase under conditions recommended by the manufacturer (Stratgene) at an annealing temperature of 54° C. The fragment was cloned into the vector PCR-Script (Stratagene) and sequenced. Sequence information for GPR 41 is provided in SEQ ID NO 5 (nucleotide and amino acid) and in SEQ ID NO: 6 (amino acid). The insert was removed as a BamHI/Xhol fragment and subcloned into the mammalian expression vector pCDNA3 (invitrogen) and yeast expression vector p426GPD.

### EXAMPLE 5

It was shown that expression of rat GPR41 (rGPR41) in HEK293T cells together with G_{o1α} also elicited carboxylic acid-mediated stimulation of [³⁵S]GTPγS binding (FIG. 5). A similar rank order of potency for C1-C5 carboxylic acids was found between human and rGPR41 suggesting similar pharmacological profiles for the two species homologues.

### EXAMPLE 6

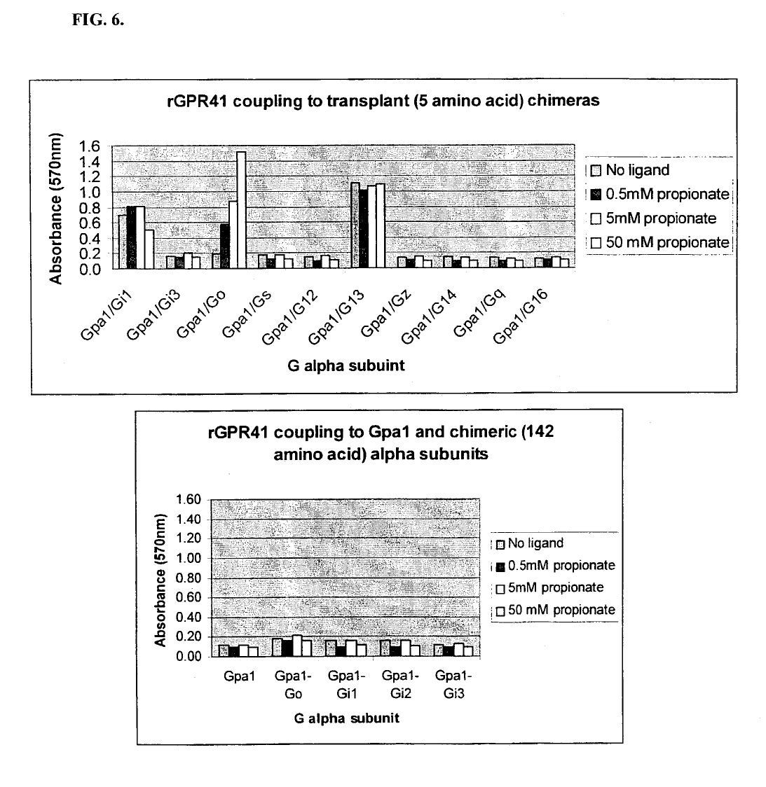

It was found that rGPR41 could be expressed in the yeast Saccharomyces cerevisiae and successfully coupled to the pheromone response pathway. Significant absorbance at 570 nm, corresponding to induction of FUS1-lacZ and FUS1-HIS3 reporter genes, was detected for MMY11 cells containing p426GPD-rGPR41 in combination with pRS314-Gpa1/G_{αo}. These cells express rGPR41 in combination with a Gα subunit identical to Gpa1 but in which the 5 C-terminal amino acids are replaced with the 5 C-terminal amino acids of the mammalian Gα subunit, G_{αo}. This response is dependant on the presence of the agonist ligand propionate since no induction is detected in the absence of propionate (FIG. 6). Control cells transformed with the vector p426GPD in combination with pRS314-Gpa1/G_{αo} and therefore lacking rGPR41 did not respond to propionate. Reporter gene activity was also detected in the presence of the Gpa1/G_{α13} transplant, but this was observed even in the absence of propionate and hence does not correspond to receptor coupling but instead to the elevated basal reporter gene expression observed previously with the Gpa1/G_{α13} protein (Brown, 1999). Of the other Gα subunits tested, the pRS314-Gpa1/G_{α12} and pRS314-Gpa1/G_{α13} transplants supported weak rGPR41 coupling detectable after 48 hrs incubation (data not shown), and with no other Gα subunit could ligand responses of rGPR41 be detected. The activation of the yeast signal transduction pathway in response to agonist, and the specificity of activation of a particular Gα subunit, confirm that the rGPR41 nucleotide sequence encodes a functional G-protein coupled receptor which can be activated by propionate. Furthermore, the specificity for Gpa1/G_{αo} combined with the weaker activation of Gpa1/G_{αi2} and pRS314-Gpa1/G_{αi3}, suggests that under physiological circumstances in mammalian cells rGPR41 is coupled to the Gi-family of G-proteins.

### EXAMPLE 7

A study was carried out of the function of rGPR41 following expression in S. cerevisiae MMY22, which is a derivative of S. cerevisiae MMY11 containing a copy of the gene encoding the Gpa1/G_{αo} transplant chimera described above (in which the 5 C-terminal amino acids of Gpa1 are replaced with the 5 C-terminal amino acids of G_{αo}) integrated into the genome. To do this, a cassette comprising the GPA1 promoter, nucleotide sequence encoding Gpa1/G_{αo}, and the terminator was sub-cloned from pRS314-Gpa1/G_{αo} into the yeast integrating plasmid pRS304 (Sikorski and Hieter, 1989). The resulting plasmid pRS304-Gpa1/G_{αo} was transformed into MMY11 and integrated into the trp1 locus creating MMY22.

The rGPR41 expression construct described above, p462GPD-rGPR41 was introduced into MMY22 by transformation as described above. Four separate transformants were tested for reporter gene activation in response to propionate. Assays for FUS1-lacZ and FUS1-HIS3 induction were performed as described, except the β-galactosidase (lacZ) substrate fluorescein di-β-D-galactopyranoside (FDG) was used in place of CPRG, and 3-aminotriazole concentration was 5 mM. Also, black-walled 96-well microtitre plates were used, and fluorescence resulting from degradation of FDG to fluorescein due to β-galactosidase was determined using a Spectrofluor microtitre plate reader (Tecan)(excitation wavelength: 485 nm; emmision wavelength: 535 nm).

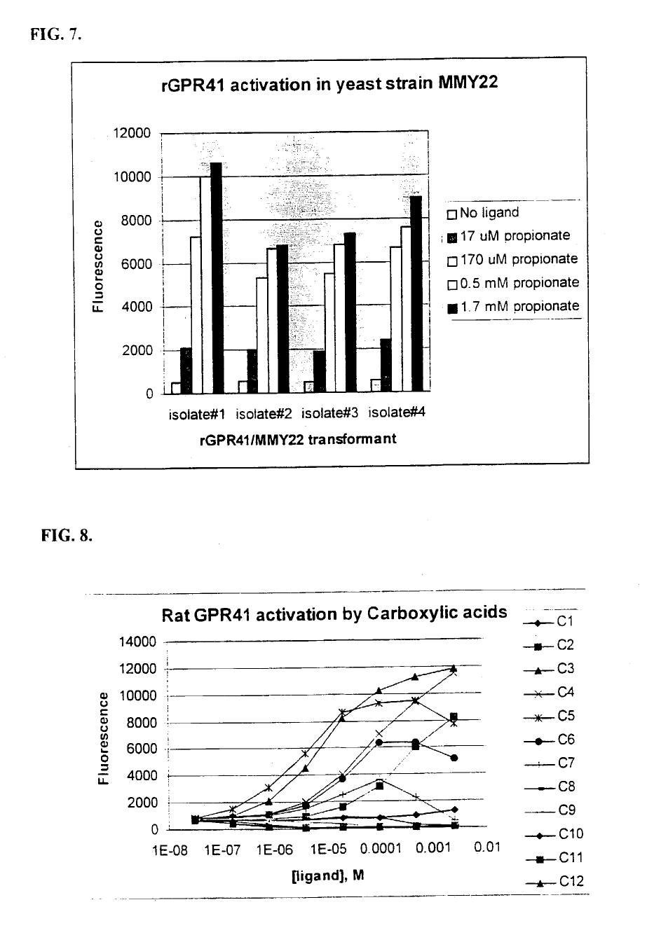

Significant fluorescence corresponding to activation of the pheromone response pathway by rGPR41 was detected. The extent of this response was dependent on the concentration of propionate, and only small basal levels of fluorescence were detected in the absence of propionate (FIG. 7). This result confirms the ability of the Gpa1/G_{αo} transplant chimera to support activation of the pheromone response pathway by rGPR41 in response to its agonist ligand. The magnitude of responses to the highest propionate concentration varied amongst transformants. This is likely to be due to small variations in copy-number of the p462GPD-rGPR41 plasmid, which are expected to vary (approximately 20 to 40 plasmid copies per cell) due to the 2 μ genetic element on this plasmid. This is likely to cause small variations in the level of rGPR41 protein.

### EXAMPLE 8

A series of carboxylic acids and other compounds related to propionate were tested for the ability to activate rGPR41. Experiments were performed as described above, using yeast cells of strain MMY22 transformed with rGPR41 expression plasmid p462GPD-rGPR41, except that the agonist propionate was replaced with one of a series of other compounds, which are listed in table 2 below.

TABLE 2
Compounds tested for ability to activate GPR 41

| | | Agonism @ |
|---|---|---|
| | | rGPR41 |
| Anion | Form tested | Approx. EC50 (M) |
| 4-pentenoate | | 3.0E−05 |
| acetamide | | very weak active |
| Acetate | Sodium acetate | 1.0E−03 |
| Acetoacetate | Lithium Acetoacetate | weak active |
| Acetone | free | Inactive |
| Alanine | free | Inactive |
| alpha-hydroxybutyrate | Sodium alpha- | Inactive |
| | hydroxybutyrate | |
| Ascorbate | Sodium ascorbate | Inactive |
| benzoate | Sodium benzoate | Toxic |
| Caproic C6 acid | Potassium salt from acid | 3.0E−05 |
| Caprylic C8 acid | Potassium salt from acid | weak active |
| Citrate | Potassium salt from acid | Inactive |
| D-b-Hydroxybutyrate | Sodium salt | 1.0E−02 |
| Decanoic C10 acid | Potassium salt from acid | inactive/toxic |
| DL-b-Hydroxybutyrate | Sodium beta- | 1.0E−02 |
| | hydroxybutyrate | |
| Formate | Ammonium formate | Weak active |
| Fumarate | | Inactive |
| GABA | | Inactive |
| γ-hydroxybutyrate | Sodium gamma- | 1.0E−02 |
| | hydroxybutyrate | |
| Glutamate | Sodium glutamate | Inactive |
| Glycine | free | Inactive |
| glycolate | | weak active |
| Glyoxal | free | Toxic |
| Glyoxylic acid | sodium glyoxylate | 5.0E−02 |
| Heptanoic C7 acid | Potassium salt from acid | 1.0E−04 |
| Isobutyrate | | 3.0E−05 |
| lactate | sodium lactate | Inactive |
| lactate | Potassium salt from acid | Inactive |
| Lauric C12 acid | Potassium salt from acid | inactive/toxic |
| L-b-Hydroxybutyrate | Sodium salt | 1.0E−02 |
| Linoleic acid | | Toxic/Inactive |
| maleate | Potassium salt from acid | Inactive |
| malonate | Sodium malonate | Inactive |
| methyl-2-imidazoline | | Toxic/inactive |
| methylphosphonate | | Inactive |
| methylsulphonate | Sodium methylsulphonate | Inactive |
| n-Butyrate | Potassium salt from acid | 5.0E−05 |
| nicotinic acid | | Inactive |
| NMDA | | Inactive |
| Nonanoic C9 acid | Potassium salt from acid | inactive/toxic |
| oleate | Sodium oleate in 1% BSA | Inactive |
| | (KOH added by mistake) | |
| Oxalate | Sodium oxalate | 3.0E−03 |
| Oxaloacetate | Potassium salt from acid | Inactive |
| palmitate | Sodium palmitate | Inactive |
| Palmitoleic acid | | Toxic/inactive |
| Pentanoate (n-valerate) | Potassium salt from acid | 3.0E−06 |
| pivalic acid | | 5.0E−04 |
| Propionate | Sodium propionate | 5.0E−06 |
| pyruvate | | Inactive |
| stearate | Sodium stearate | Inactive |
| Succinate | disodium succinate | Inactive |
| Tartrate | Sodium tartrate | Inactive |
| tiglic acid | | Inactive |
| Trichloroacetate | Potassium salt from acid | Toxic |
| Trifluoroacetate | Na TFA | weak active |
| Undecanoic C11 acid | Potassium salt from acid | inactive/toxic |

The compounds were generally organic anions, which were introduced into the assay as sodium or potassium salt solutions buffered to pH 7.0. The extent of GPR 41 activation due to the compound tested is also shown in Table 2. Where significant activity was detected, the relative activity is given as approximate EC50 value, where propionate gave EC50 value of 5 μM. The most active compounds tested were unsaturated, straight or branched chain carboxylic acids (short-chain fatty acids). Concentration-response curves for the series of unsaturated, straight-chain carboxylic acids containing from 1 to 12 carbon atoms is shown in FIG. 8. Pentanoate (n-valerate), the carboxylic acid containing five carbon atoms, is similarly or slightly more active than propionate. The order of agonist potency of this series of ligands is: C1<C2<C3=C5>C6>C7>C8, also C3>C4<C5, and C9, C10, C11 and C12 are inactive, where C1=formate, C2=acetate, C3=propionate, C4=butyrate, C5=pentanoate, C6=caproate, C7=heptanoate, C8=caprylate, C9=nonanoate, C10=decanoate, C11=undecanoate, and C12=dodecanoate. This experiment demonstrates that positions of hydrogen atoms within a canonical straight chain, unsaturated carboxylic acid agonist (such as propionate, butyrate, or pentanoate) may be substituted for hydroxyl groups or fluorine atoms without abolishing activity, though not larger halogens such as chlorine. Substitution with methyl groups giving branched chains (as in isobutyric acid and pivalic acid) or carbon-carbon double bonds giving unsaturated chains (as in 4-pentenoate) also may be tolerated without abolishing activity. The carboxylic acid group appears to be required for activity. A series of naturally occurring compounds containing multiple carboxylic acids (succinate, fumarate, citrate, etc) were either inactive or had very weak activities.

### EXAMPLE 9

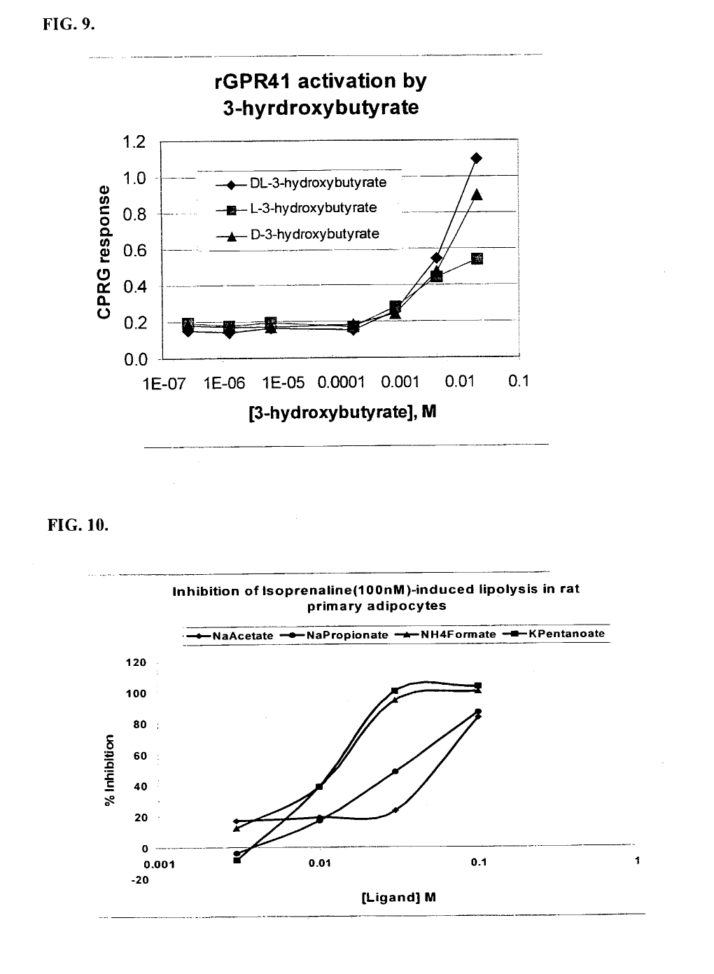

A study was made of the agonism of rGPR41 by the naturally-occurring ketone body compound, 3-hydroxybutyrate. Experiments were performed as described above, using yeast cells of strain MMY22 transformed with rGPR41 expression plasmid p462GPD-rGPR41, except that the substrate CPRG was used. Also, the agonist propionate was replaced with 3-hydroxybutyrate (β-hydroxybutyrate), either as a racemic mix of both stereoisomers, or as purified enantiomers D-3-hydroxybutyrate, or L-3-hydroxybutyrate. 3-hydroxybutyrate acted as an agonist at rGPR41 with a potency similar or slightly less than acetate (FIG. 9). The lowest concentrations of β-hydroxybutyrate which caused detectable activation of rGPR41 were in the range 0.1 mM to 1 mM. The stereoisomers had broadly similar activities: D-3-hydroxybutyrate was similarly active to the racemic mix DL-3-hydroxybutyrate, and possibly slightly more active at the highest concentration tested than L-3-hydroxybutyrate.

### EXAMPLE 10

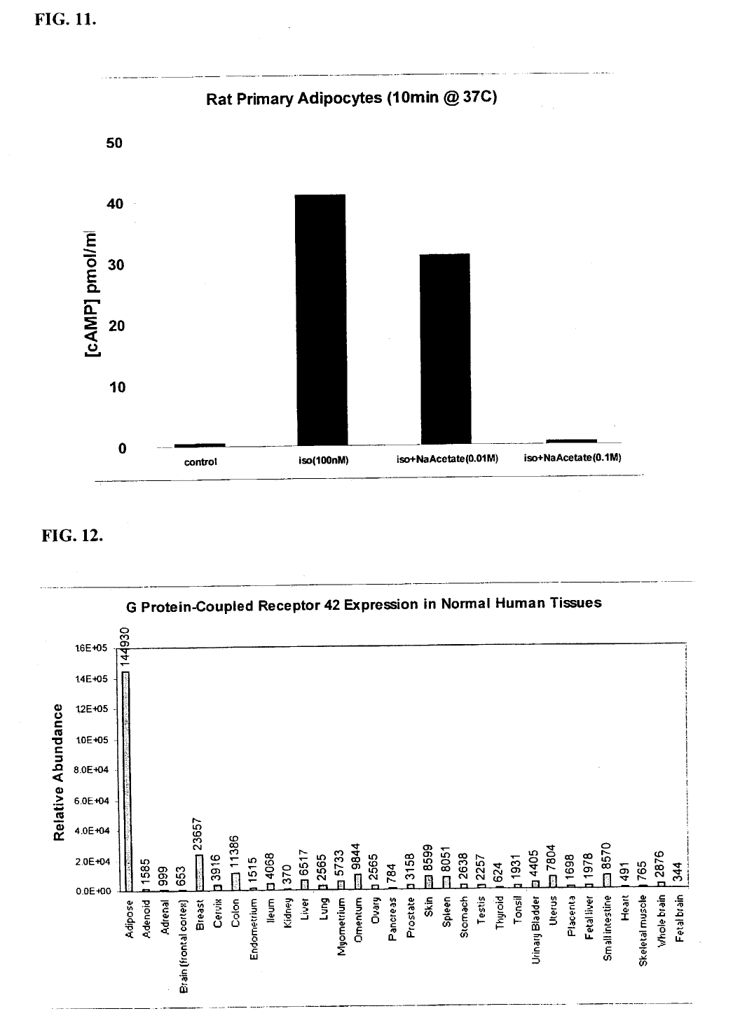

Carboxylic acids were tested for the ability to alter Gᵢ-mediated signalling mechanisms in adipocytes. Application of C1-C4 to freshly isolated primary adipocytes from rat eididymal fat pads led to significant inhibition of isoprenaline-stimulated lipolysis in a concentration-dependent manner (FIG. 10). We also demonstrated that over a similar concentration range acetate (C2) also caused a reduction of isoprenaline-amplified cAMP levels (FIG. 11). Isoprenaline stimulates adenylate cyclase activity and subsequently the process of lipolysis following activation of β-adrenoceptors and G,a in adipocytes. Hence, inhibition of these activities is normally via receptor-mediated stimulation of Gᵢ G protein signalling. These data therefore demonstrate that carboxylic acids which are ligands at GPR41, a receptor which is highly expressed in adipose tissue, can regulate Gᵢ G protein signalling pathways mediated possibly via direct activation of GPR41.

Of the various compounds acting as GPR 41 agonists identified from screening the ketone body compound 3-hydroxyutyrate is of particular interest as a GPR 41 agonist, as it is known to occur physiologically in blood, and may have effects in adipocyte lipolysis. The presence of GPR 41, a receptor responsive to 3-hydroxybutyrate on adipocytes, suggests that GPR 41 may mediate 3-hydroxybutyrate induced inhibition of lipolysis and/or increased adipocyte sensitivity to insulin. A role for GPR 41 in regulating lipolysis is further supported by demonstration that this receptor is Gi coupled and is therefore expected to inhibit adenylate cyclase on activation, since the lipase responsible for lipolysis is regulated by cAMP levels. Also in heterologous assays described above, the sensitivity of GPR 41 to 3-hydroxybutyrate is in the physiological concentration range, and further more the threshold of GPR 41 activation occurs at very close to levels which would cause acidosis (approximately 1 mM see FIG. 9).

Methods

Mammalian Cell Culture and Transfections

HEK293T cells (HEK293 cells stably expressing the SV40 large T-antigen) were maintained in DMEM containing 10% (v/v) foetal calf serum and 2 mM glutamine. Cells were seeded in 60 mm culture dishes and grown to 60-80% confluency (18-24 h) prior to transfection with pCDNA3 containing the relevant DNA species using Lipofectamine reagent. For transfection, 3 μg of DNA was mixed with 10 μl of Lipofectamine in 0.2 ml of Opti-MEM (Life Technologies Inc.) and was incubated at room temperature for 30 min prior to the addition of 1.6 ml of Opti-MEM. Cells were exposed to the Lipofectamine/DNA mixture for 5 h and 2 ml of 20% (v/v) newborn calf serum in DMEM was then added. Cells were harvested 48-72 h after transfection.

Preparation of Membranes

Plasma membrane-containing P2 particulate fractions were prepared from cell pastes frozen at −80° C. after harvest. All procedures were carried out at 4° C. Cell pellets were resuspended in 1 ml of 10 mM Tris-HCl and 0.1 mM EDTA, pH 7.5 (buffer A) and by homogenisation for 20 s with a polytron homogeniser followed by passage (5 times) through a 25-guage needle. Cell lysates were centrifuged at 1,000 g for 10 min in a microcentrifuge to pellet the nuclei and unbroken cells and P2 particulate fractions were recovered by microcentrifugation at 16,000 g for 30 min. P2 particulate fractions were resuspended in buffer A and stored at −80° C. until required. Protein concentrations were determined using the bicinchoninic acid (BCA) procedure (4) using BSA as a standard.

High Affinity [³⁵S]GTPγS Binding

Assays were performed in 96-well format using a method modified from Wieland and Jakobs, 1994. Membranes (10 μg per point) were diluted to 0.083 mg/ml in assay buffer (20 mM HEPES, 100 mM NaCl, 10 mM MgCl₂, pH7.4) supplemented with saponin (10 mg/l) and pre-incubated with 40 μM GDP. Various concentrations of nicotinic acid were added, followed by [³⁵S]GTPγS (1170 Ci/mmol, Amersham) at 0.3 nM (total vol. of 100 μl) and binding was allowed to proceed at room temperature for 30 min. Non-specific binding was determined by the inclusion of 0.6 mM GTP. Wheatgerm agglutinin SPA beads (Amersham) (0.5 mg) in 25 μl assay buffer were added and the whole was incubated at room temperature for 30 min with agitation. Plates were centrifuged at 1500 g for 5 min and bound [³⁵S]GTPγS was determined by scintillation counting on a Wallac 1450 microbeta Trilux scintillation counter.

Construction of p426GPD-rGPR41 for Expression of rGPR41 in Yeast Cells

Nucleotide sequence encoding rGPR41 flanked by restriction enzyme sites BamHI and XhoI was cloned into the yeast expression vector p426GPD (Mumberg, 1995). The orientation of insertion was such that, when introduced into yeast cells, transcription from the GPD promoter contained within the p426GPD-rGPR41 plasmid resulted in production of mRNA encoding rGPR41 protein. The GPD promoter sequence in p426GPD is a copy of the chromosomal sequence upstream of the highly expressed yeast gene, TDH1. Hence, yeast cells containing p426GPD-rGPR41 should produce rGPR41 protein to high levels.

Transformation of Yeast Strain MMY11 with Construct p426GPD-rGPR41 in Combination with Gα Subunit Expression Constructs

The yeast strain MMY11 has been described previously (Olesnicky et al, 1999). It contains a series of genetic modifications to enable coupling of heterologously expressed receptors to the expression of two reporter genes, via the endogenous yeast pheromone response signal transduction pathway. Importantly, the gene encoding the endogenous yeast pheromone receptor, STE2, has been deleted from MMY1 1 such that cells of strain MMY11 containing p426GPD-rGPR41 will express rGPR41 protein in place of Ste2 receptor protein. Furthermore, the gene encoding the G-protein α-subunit involved in the pheromone response, GPA1, has been deleted from MMY11. To enable receptor coupling in strain MMY11, plasmid constructs encoding either wild-type GPA1 of modified versions of GPA1 are introduced into MMY11 and are expressed in place of endogenous yeast GPA1. The series of plasmids encoding modified versions of GPA1 has been described previously (Brown et al., 1999) and is the subject of patent (application number PCT.GB98.02759). Generally, the modifications made to Gpa1 facilitate coupling of heterologously expressed receptors to the yeast pheromone response pathway. Yeast strain MMY11 was transformed with pairs of plasmids, the first being p426GPD-rGPR41 and the second being one of the pRS314-based Gα expression constructs from table 3 below. Yeast transformations were performed according to the routine methods (Gietz et al., 1992).

TABLE 3
pRS314-based Gα expression constructs used in this experiment:

| | Modification to Gpal C- | Nature of Gα |
|---|---|---|
| Gα expression construct | terminus | subunit |
| pRS314-Gpal | None | Wild-type Gpal |
| pRS314-Gpal/G_{αq} | 5 amino acids replaced | Chimera (transplant) |
| pRS314-Gpal/G_{αs} | 5 amino acids replaced | Chimera (transplant) |
| pRS314-Gpal/G_{αo} | 5 amino acids replaced | Chimera (transplant) |
| pRS314-Gpal/G_{αi2} | 5 amino acids replaced | Chimera (transplant) |
| pRS314-Gpal/G_{αi3} | 5 amino acids replaced | Chimera (transplant) |
| pRS314-Gpal/G_{αz} | 5 amino acids replaced | Chimera (transplant) |
| pRS314-Gpal/G_{α12} | 5 amino acids replaced | Chimera (transplant) |
| pRS314-Gpal/G_{α13} | 5 amino acids replaced | Chimera (transplant) |
| pRS314-Gpal/G_{α14} | 5 amino acids replaced | Chimera (transplant) |
| pRS314-Gpal/G_{α16} | 5 amino acids replaced | Chimera (transplant) |
| pRS314-Gpal-G_{αo} | 142 amino acids replaced | Chimera |
| pRS314-Gpal-G_{αi1} | 142 amino acids replaced | Chimera |
| pRS314-Gpal-G_{αi2} | 142 amino acids replaced | Chimera |
| pRS314-Gpal/G_{αi3} | 142 amino acids replaced | Chimera |

Assay for Induction of Reporter Genes FUS1-lacZ and FUS1-HIS3 in Response to GPR41 Ligands

In vivo assays of reporter gene induction were carried out by suspending MMY11 cells transformed as described above to a density of 0.02 OD₆₀₀/ml in 200 μl SC-glucose (2%) medium lacking tryptophan, uracil and histidine. This medium was supplemented with 10 mM 3-aminotriazole and the β-galactosidase (lacZ) substrate chlorophenolred-β-D-galactopyranoside (CPRG; Boehringer Mannheim) to a concentration of 0.1 mg/ml. Additionally the medium was supplemented with various concentrations of the agonist ligand, propionate (sodium propionate, pH 7.0). To visualise the yellow to red colour change reaction occurring on degradation of CPRG due to β-galactosidase, the medium was buffered to pH 7 with 0.1 M sodium phosphate. The assay was conducted in flat-bottomed sterile 96-well microtitre plates. Plates were incubated for 24 hours at 30° C. without agitation, and absorbance at 570 nm was determined using a Spectrofluor microtitre plate reader (Tecan).

Preparation of Rat Primary Adipocytes

70 ml of the “collection buffer” is prepared freshly each day. This buffer consists of 2.8 g of BSA (Sigma: A 7030) dissolved in 70 ml of DMEM (HEPES modification, Sigma: D6171), to aid the dissolution of the BSA the media is incubated at 37° C. Approximately 30 ml of the buffer were then transferred to a 70 ml Sterilin pot for the collection of rat epididymal fat pads. The remainder of the buffer is used to prepare the collagenase solution, which is freshly prepared for each experiment. Collagenase Type II (25 mg; Sigma: C 6885) is dissolved in to 25 ml of the “collection buffer” and supplemented with 80 μl of a 1M CaCl₂ solution (to give a final Ca²⁺ concentration of 5 mM) and 25 μl of adenosine deaminase (Sigma: A 1030; final concentration=10 μg/ml).

The freshly removed fat pads are then individually weighed and then cut in to small pieces and each fat pad are added to a 50 ml conical flask containing 12.5 ml of the 1 mg/ml collagenase solution. No more than 6 grams, wet weight, of adipose tissue is added to each 12.5 ml volume of collagenase solution. The adipose tissue is then incubated for 60-75 minutes at 37° C. whilst being mixed at 150 cycles per minute. At the end of the incubation period the adipose tissue is filtered through a 100 μm mesh (Falcon) in to a 50 ml Falcon tube. In order to facilitate the passage of the adipocytes through the filter they are flushed with Krebs buffer which has been supplemented with BSA (1%) and ADA (10 μg/ml).

The adipocytes are then centrifuged (500 rpm, 1 minute) to allow the adipocytes to float to the surface. The infranatant is removed, and the volume made up to 35 ml by the addition of fresh Krebs Buffer. The adipocytes were again centrifuged (500 rpm, 1 minute) and the infranatant removed. This washing step is repeated and the residual adipocytes transferred to a 5ml Sterilin tube and kept at 37° C. prior to use.

Adipocyte Lipolysis Assays

The lipolysis assay was performed on a 24-well plate, in a volume of 1 ml. 800 μl of Krebs buffer was added in to each well. Test compounds or their vehicle was added as 100-fold concentrated stocks (i.e. 10 μl per well). Isoprenaline (100 μl of a 1 μM solution) was added to the relevant wells to give a final concentration of 100 nM. Finally the assay was started by the addition of 100 μl of the adipocyte suspension. The 24-well plate was then transferred to an incubator (37° C./5% CO₂) and left for 2 hours At the end of this incubation period, a 25 μl samples were removed from each well and transferred to a 96-well plate. The levels of glycerol were then determined by a commercially available assay (Randox).

In this test system several carboxylic acid derivatives were tested up to concentrations of 0.1 M. A concentration-related inhibition of lipolysis was observed with these ligands. Inhibition of glycerol was not due to a direct effect in the assay since, in a no adipocyte control, none of the compounds tested effected the assay at concentrations up to 1 M.

cAMP Quantification

A reaction mixture of 500 μl was used in a 1.5 ml eppendorf tube. The reaction was started by the addition of 100 μl of adipocytes to a 400 μl volume of Krebs buffer containing isoprenaline and test compound. Following the addition of the adipocytes the reaction tube was incubated at 37° C. for 10 minutes. The reaction was stopped with the addition of 500 μl of a stop mixture containing methanol (1 part), chloroform (2 parts) and 0.1N HCl (0.1 part). Following the addition of the stop mixture each tube was vortexed and then centifuged at 5000 rpm for 5 minutes. 300 μl of the supernatent was removed and stored at −20° C. The levels of cAMP in each samples were determined using an ELISA kit from R&D systems (DE0355).

1. A method for identification of an agent that modulates activity of G-protein coupled receptor 41 (GPR 41), or G-protein coupled receptor 42 (GPR 42) which method comprises:
(i) contacting a test agent with GPR 41, GPR 42 or a variant of either thereof which is capable of coupling to a G-protein; and
(ii) monitoring for GPR 41 or GPR 42 activity in the presence of a G-protein;
thereby determining whether the test agent modulates GPR 41 or GPR 42 activity.

2. A method according claim I wherein the test agent is contacted in step (i) with cells that express GPR 41, GPR 42 or a said variant of either thereof.

3. A method according to claim I wherein the test agent is contacted in step (i) with the membrane of cells that express GPR 41, GPR 42 or a said variant of either thereof.

4. A method according to claim 2 or 3 wherein the cells are adipocytes.

5. A method according to claim 4 wherein the adipocytes are provided as a differentiated cell line.

6. A method according to claim 4 wherein the adipocytes are primary adipocytes harvested from a human or animal donor.

7. A method according to any one of the preceding claims wherein the variant has at least 70% sequence identity to SEQ ID NO: 2 or SEQ ID NO: 4.

8. A method according to any one of the preceding claims wherein the G-protein is Gᵢ-protein.

9. A method according to claim 8 wherein step (ii) comprises determining whether Gᵢ-protein is activated.

10. A test kit suitable for identification of an agent that modulates GPR 41 or GPR 42 activity, which kit comprises:
(a) GPR 41, GPR 42 or a variant of either thereof which is capable of coupling to a Gᵢ-protein; and
(b) means for monitoring GPR 41 or GPR 42 activity.

11. A kit according to claim 10 wherein component (a) comprises cells which express GPR 41, GPR 42 or a said variant of either thereof.

12. A kit according to claims 10 or 11 wherein component (b) comprises means for determining whether Gᵢ-protein is activated.

13. A method for identification of an agent that inhibits lipolysis, which method comprises contacting adipocytes in vitro with a test agent identified by the method of any one of claims 1 to 9 and monitoring lipolysis, thereby determining whether the test agent is an inhibitor of lipolysis.

14. An activator of GPR 41 or GPR 42 activity identified by a method according to any one of claims 1 or 9, an inhibitor of lipolysis identified by a method according to claim 13 or a polynucleotide which encodes GPR 41, GPR 42 or a variant polypeptide of either thereof as defined in claim 1, for use in a method of treatment of the human or animal body by therapy.

15. An activator, inhibitor or polynucleotide according to claim 14 for use in the treatment of dyslipidaemia, coronary heart disease, atherosclerosis, thrombosis or obesity, angina, chronic renal failure, peripheral vascular disease, stroke, type II diabetes or metabolic syndrome (syndrome X).

16. A polynucleotide according to claim 14 or 15 comprising
(a) the nucleotide sequence of SEQ ID NO: 1 or SEQ ID NO: 3,
(b) a sequence which hybridizes under stringent conditions to the complement of SEQ ID NO: 1 or SEQ ID NO: 3,
(c) a sequence that is degenerate as a result of the genetic code with respect to a sequence defined in (a) or (b), or
(d) a sequence having at least 60% identity to a sequence as defined (a), (b) or (c).

17. Use of an activator, inhibitor or polynucleotide as defined in claim 14 in the manufacture of a medicament for the treatment of dyslipidaemia, coronary heart disease, atherosclerosis, thrombosis or obesity, angina, chronic renal failure, peripheral vascular disease, stroke, type II diabetes or metabolic syndrome (syndrome X).

18. An activator of GPR 41 or GPR 42 activity or a polynucleotide which encodes GPR 41, GPR 42 or a variant polypeptide of either thereof as defined in claim 1, for use in the treatment of dyslipidaemia, coronary heart disease, atherosclerosis, thrombosis or obesity, angina, chronic renal failure, penpteral; vascular disease, stroke, type II diabetes or metabolic syndrome (syndrome X).
